(19) United States
(12) Patent Application Publication
(10) Pub. No.: US 2009/0209638 A1
(43) Pub. Date: Aug. 20, 2009

(54) TREATMENT FOR DEPRESSIVE DISORDERS

(76) Inventors: Gunther Birznieks, Bethesda, MD (US); Deepak Phadke, Olathe, KS (US); Mihael H. Polymeropoulos, Potomac, MD (US)

Correspondence Address: HOFFMAN WARNICK LLC, 75 STATE STREET, 14TH FLOOR, ALBANY, NY, 12207, US

(21) Appl. No.: 12/301,668

(22) PCT Filed: May 22, 2007
(86) PCT No.: PCT/US07/69420

§ 371 Date: Nov. 20, 2008

Related U.S. Application Data
(60) Provisional application No. 60/747,843, filed on May 22, 2006.

(51) Int. Cl. A61K 31/343; A61P 25/24

(52) U.S. Cl. 514/469

(57) ABSTRACT

A method of treating depression comprising administering a melatonin agonist.

### CROSS-REFERENCE TO RELATED APPLICATIONS

This application claims the benefit of co-pending U.S. Provisional Patent Application No. 60/747,843, filed 22 May 2006, which is hereby incorporated herein.

### BACKGROUND OF THE INVENTION

1. Field of the Invention

This invention is in the field of drug therapy for depressive illnesses.

2. Related Art

Depressive disorders affect nearly 20 million adults in the U.S. alone. Left untreated, depressive disorders can be debilitating, emotionally as well as physically.

Depressive disorders comprise an array of symptoms, which are listed in a booklet published by the U.S. National Institute of Mental Health (NIMH), entitled, “Depression,” as follows:

“Persistent sad, anxious, or “empty” mood

Feelings of hopelessness, pessimism

Feelings of guilt, worthlessness, helplessness

Loss of interest or pleasure in hobbies and activities that were once enjoyed, including sex

Decreased energy, fatigue, being “slowed down”

Difficulty concentrating, remembering, making decisions

Insomnia, early-morning awakening, or oversleeping

Appetite and/or weight loss or overeating and weight gain

Thoughts of death or suicide; suicide attempts

Restlessness, irritability

Persistent physical symptoms that do not respond to treatment, such as headaches, digestive disorders, and chronic pain.”

According to the NIMH booklet, three of the most common types of depressive illness are:

“Major depression is manifested by a combination of symptoms (see symptom list) that interfere with the ability to work, study, sleep, eat, and enjoy once pleasurable activities. Such a disabling episode of depression may occur only once but more commonly occurs several times in a lifetime.

A less severe type of depression, dysthymia, involves long-term, chronic symptoms that do not disable, but keep one from functioning well or from feeling good. Many people with dysthymia also experience major depressive episodes at some time in their lives.

Another type of depression is bipolar disorder, also called manic-depressive illness. Not nearly as prevalent as other forms of depressive disorders, bipolar disorder is characterized by cycling mood changes: severe highs (mania) and lows (depression). Sometimes the mood switches are dramatic and rapid, but most often they are gradual. When in the depressed cycle, an individual can have any or all of the symptoms of a depressive disorder. When in the manic cycle, the individual may be overactive, overtalkative, and have a great deal of energy. Mania often affects thinking, judgment, and social behavior in ways that cause serious problems and embarrassment. For example, the individual in a manic phase may feel elated, full of grand schemes that might range from unwise business decisions to romantic sprees. Mania, left untreated, may worsen to a psychotic state.”

The compound referred to herein as MA-1 is (1R-trans)-N-[[2-(2,3-dihydro-4-benzofuranyl)cyclopropyl]methyl]propanamide. It is an experimental melatonergic agonist that has high affinity for both the Melatonin-1 (MT1) and Melatonin-2 (MT2) receptors and is therefore potentially useful for the treatment of insomnia and circadian rhythm sleep disorders. MA-1 is disclosed in U.S. Pat. No. 5,856,529, which is incorporated by reference herein as though fully set forth. The compound referred to herein as MA-2 is N-[[2-(2,3-dihydro-4benzofuranyl)cyclo-propyl]methyl]propanamide (herein referred to as MA-1), N-[1-(2,3-dihydrobenzofuran-4-yl)pyrrolidin-3-yl]-N-ethylurea]. It is also an experimental melatonergic agonist and is disclosed in U.S. Pat. No. 6,211,225, which is incorporated by reference herein as though fully set forth.

### SUMMARY OF THE INVENTION

The method of the invention comprises treatment of one or more depressive disorders in an animal, as well as the treatment of one or more symptoms of a depressive illness.

The method of the invention also comprises treatment or prevention of other disorders for which certain antidepressants, e.g., serotonin reuptake inhibitors, have been shown to be useful. These include but are not limited to obsessive-compulsive disorder, panic disorder, social anxiety disorder, social phobia, post-traumatic stress disorder, premenstrual dysphoric disorder, and generalized anxiety disorder.

### DETAILED DESCRIPTION

This invention, which is hereinafter described with respect to illustrative embodiments, contemplates use of the melatonin agonists herein referred to as MA-1 and MA-2, including salts, prodrugs, esters, metabolites, solvates, hydrates, enantiomers, stereoisomers, and amorphous and crystalline forms thereof. MA-1 is a white to off-white powder with a melting point of about 78° C. (DSC) and has the structure illustrated in Formula 1.

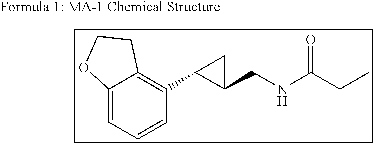

Metabolites of MA-1 include, for example, those described in “Preclinical Pharmacokinetics and Metabolism of BMS-214778, a Novel Melatonin Receptor Agonist” by Vachharajani et al., J. Pharmaceutical Sci., 92(4):760-772, which is hereby incorporated herein by reference. More specifically, these metabolites include hydroxylated and dehydrogenated derivatives of MA-1 as well as glucuronide and diol derivatives of MA-1. The structures of eight such metabolites have Formulae 2-9.

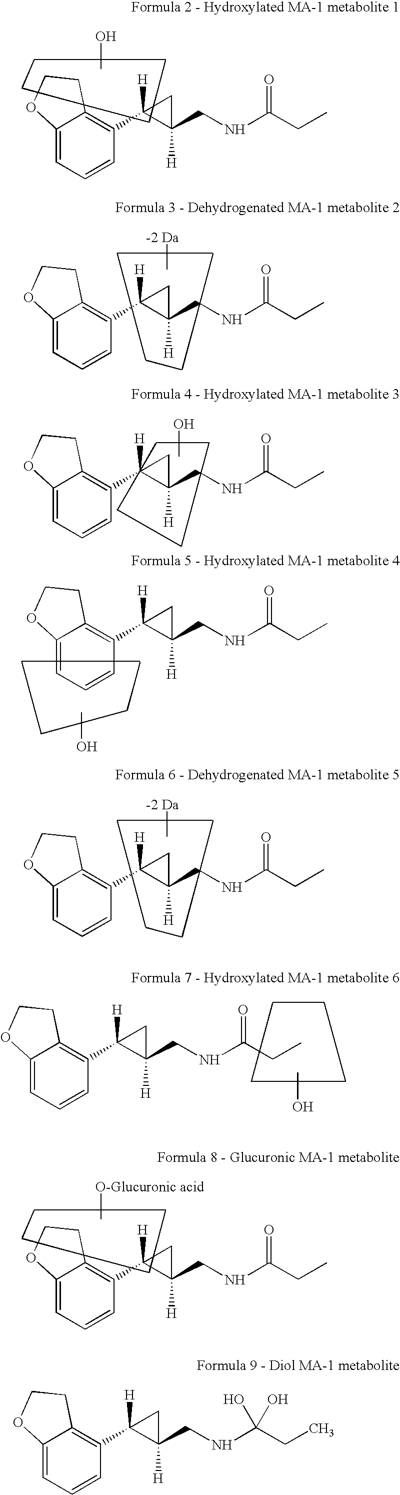

An effective amount of MA-1 or MA-2 may be administered to a subject animal (typically a human but other animals, e.g., farm animals, pets and racing animals, can also be treated) by a number of routes. An effective amount is an amount that during the course of therapy will have a preventive or ameliorative effect on a depressive disorder or a symptom thereof. For example, an effective amount is an amount that prevents the occurrence or recurrence of symptoms of a depressive disorder to the same degree as other antidepressants, e.g., selective serotonin re-uptake inhibitors such as fluoxetine, paroxetine, sertraline, etc.

An effective amount, quantitatively, may vary, e.g., depending upon the patient, the severity of the disorder or symptom being treated, and the route of administration. Such dose can be determined by routine studies. In general, for systemic administration, e.g., oral administration, a reference point for dosing is the dose of a MA-1 or MA-2 that is used to treat circadian rhythm disorders in humans, i.e., 1 to 500 mg/day when administered orally. It is expected that MA-1 or MA-2 can be administered to adult humans at doses of 1 to 500 mg/day, although to avoid possible adverse events, it is preferable to use lower doses, e.g., 150, 100, 50, 25, 10 or 1 mg/day. In general, the dose of MA-1 will be in the range of about 10 to about 150 mg/day, preferably, about 10 to about 100 mg/day, in one or more unit dosage forms.

It will be understood that the dosing protocol including the amount of MA-1 or MA-2 actually administered will be determined by a physician in the light of the relevant circumstances including, for example, the condition to be treated, the chosen route of administration, the age, weight, and response of the individual patient, and the severity of the patient's symptoms. Patients should of course be monitored for possible adverse events.

For therapeutic or prophylactic use, MA-1 or MA-2 will normally be administered as a pharmaceutical composition comprising as the (or an) essential active ingredient at least one such compound in association with a solid or liquid pharmaceutically acceptable carrier and, optionally, with pharmaceutically acceptable adjuvants and excipients employing standard and conventional techniques.

MA-1 is very soluble or freely soluble in 95% ethanol, methanol, acetonitrile, ethyl acetate, isopropanol, polyethylene glycols (PEG-300 and PEG-400), and only slightly soluble in water. The native pH of a saturated solution of MA-1 in water is 8.5 and its aqueous solubility is practically unaffected by pH.

Pharmaceutical compositions useful in the practice of this invention include suitable dosage forms for oral, parenteral (including subcutaneous, intramuscular, intradermal and intravenous), transdermal, bronchial or nasal administration. Thus, if a solid carrier is used, the preparation may be tableted, placed in a hard gelatin capsule in powder or pellet form, or in the form of a troche or lozenge. The solid carrier may contain conventional excipients such as binding agents, fillers, tableting lubricants, disintegrants, wetting agents and the like. The tablet may, if desired, be film coated by conventional techniques. If a liquid carrier is employed, the preparation may be in the form of a syrup, emulsion, soft gelatin capsule, sterile vehicle for injection, an aqueous or non-aqueous liquid suspension, or may be a dry product for reconstitution with water or other suitable vehicle before use. Liquid preparations may contain conventional additives such as suspending agents, emulsifying agents, wetting agents, non-aqueous vehicle (including edible oils), preservatives, as well as flavoring and/or coloring agents. For parenteral administration, a vehicle normally will comprise sterile water, at least in large part, although saline solutions, glucose solutions and like may be utilized. Injectable suspensions also may be used, in which case conventional suspending agents may be employed. Conventional preservatives, buffering agents and the like also may be added to the parenteral dosage forms. Particularly useful is the administration of a compound of Formula I in oral dosage formulations. The pharmaceutical compositions may be prepared by conventional techniques appropriate to the desired preparation containing appropriate amounts of MA-1 or MA-2. See, for example, Remington's Pharmaceutical Sciences, Mack Publishing Company, Easton, Pa., 17th edition, 1985.

In making pharmaceutical compositions for use in the invention, the active ingredient(s) will usually be mixed with a carrier, or diluted by a carrier, or enclosed within a carrier which may be in the form of a capsule, sachet, paper or other container. When the carrier serves as a diluent, it may be a solid, semi-solid or liquid material which acts as a vehicle, excipient or medium for the active ingredient. Thus, the composition can be in the form of tablets, pills, powders, lozenges, sachets, cachets, elixirs, suspensions, emulsions, solutions, syrups, aerosols (as a solid or in a liquid medium), ointments containing for example up to 10% by weight of the active compound, soft and hard gelatin capsules, suppositories, sterile injectable solutions and sterile packaged powders.

Some examples of suitable carriers and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, methyl cellulose, methyl- and propylhydroxybenzoates, talc, magnesium stearate and mineral oil. The formulations can additionally include lubricating agents, wetting agents, emulsifying and suspending agents, preserving agents, sweetening agents, or flavoring agents. The compositions of the invention may be formulated so as to provide quick, sustained, or delayed release of the active ingredient after administration to the patient.

The compositions are preferably formulated in a unit dosage form, each dosage containing from about 0.1 to about 100 mg of the active ingredient. The term “unit dosage form” refers to physically discrete units suitable as unitary dosages for human subjects and other mammals, each unit containing a predetermined quantity of active material calculated to produce the desired prophylactic or therapeutic effect over the course of a treatment period, in association with the required pharmaceutical carrier. So, for example, an adult patient suffering a depressive disorder could be prescribed 1-4 tablets, each having 10-100 mg of MA-1, to be taken once, twice or three times daily and might expect improvement in his or her condition within about one to about 12 weeks.

A typical unit dose form could be size 0 or size 1 capsule comprising 10, 20, 50, or 100 mg of MA-1 in addition to anhydrous lactose, microcrystalline cellulose, silicon dioxide colloidal, croscarmellose sodium, and magnesium stearate. Storage at 15 to 20° C. with protection from moisture and sunlight is recommended.

MA-1 can also be formulated in a controlled release form, e.g., delayed, sustained, or pulsatile release. MA-1 can also be administered concomitantly with other drug therapies, including but not limited to other antidepressant drug therapies or other drug therapies for treating other emotional disorders. So, for example, the invention encompasses administration of MA-1 or MA-2 in combination with other melatonergic agonists or other sleep-inducing agents. Other antidepressant agents include, but are not limited to, agents in the following drug categories:

melatonin agonists

selective serotonin reuptake inhibitors (SSRIs)
5-HT_{1A} antagonists
5-HT_{1A}/β-adrenoceptor antagonist
5-HT_{1B} antagonists
5-HT_{2C} antagonists
   Selective and nonselective
5-HT_{2C} agonists
5-HT₆ agonists
α-2 adrenergic antagonists

serotonin and norepinephrine reuptake inhibitors (SNRIs)

monoamine oxidase inhibitors (MAOIs)

tricyclic antidepressants (TCAs)

triple monoamine update blockers

benzodiazepines

NMDA receptor antagonists

Pyrrolinones

Benzothiadiazides

Benzoylpiperidnes

Biarylopropylsulfonamides

Metabotropic glutamate receptors (mGluRs)

GABA antagonists

NK1 antagonists

NK2 antagonists

CRF1 antagonists

Arginine vasopressin V1b antagonists

MCH receptor antagonists

NGF antagonists

BDNF antagonists

NT-3 antagonists

NT-4 antagonists

CREB antagonists

Illustrative, and not limiting, of such agents are:

melatonergic agonists: melatonin, agomelatine, (1R-Trans)-N-[[2-(2,3-dihydro-4-benzofuranyl)cyclopropyl]methyl]propan-amide, and N-[1-(2,3-dihydrobenzofuran-4-yl)pyrrolidin-3-yl]-N-ethylurea], ramelteon, 2-Phenylmelatonin, 8-M-PDOT, 2-Iodomelatonin, 6-Chloromelatonin;

serotonin reuptake inhibitors: paroxetine, fluoxetine, sertraline, venlaxafine, citalopram, escitalopram, fluvoxamine, trazadone, nefazodone, milnacipran, desipramine, duloxetine, YM992;

SSRI/5-HT1A antagonists: WAY-100635, Pindolol;

SSRI/5-HT1B antagonists: SB-224289;

SSRI/5-HT2C antagonists;

Selective: SB242084, RS102221;

Nonselective: Ketanserin, Irindalone;

SSRI/5-HT2C agonists: Org 37684, Ro 60-0175, WAY-161503, YM348, WAY-629, WAY-1 63909;

SSRI/5-HT6 agonists: LY586713, WAY-466, WAY-1811187;

α-2 adrenergic antagonists: Mirtazapine (Remeron);

triple monoamine update blockers: DOV 21,947;

NMDA receptor antagonists: MK-801, Memantine, Ketamine, Felbamate, Glycine, D-serine, D-cycloserine, L-glutamatelfenprodil;

Pyrrolidiones: Piracetam, Aniracetam;

tricyclics: Amitriptyline Clomipramine Desipramine Dothiepin Doxepin Imipramine Lofepramine Nortriptyline Protriptyline Trimipramine Iprindole Opipramol;

tetracyclics: Maprotiline, Mianserin, Mirtazapine, Amoxapine, Trazodone, Nefazodone;

serotonin reuptake enhancers: tianeptine;

monoamine oxidase inhibitors: Harmaline Nialamide Selegiline Isocarboxazid Iproniazid Iproclozide Moclobemide Phenelzine Toloxatone Tranylcypromine;

dopamine reuptake inhibitors: Bupropion Amineptine Methylphenidate Phenmetrazine Vanoxerine;

norepinephrine reuptake inhibitors: Atomoxetine Reboxetine Viloxazine Maprotiline Bupropion, Reboxetine;

serotonin-norepinephrine reuptake inhibitors: Desipramine Duloxetine Milnacipran Nefazodone Venlafaxine;

Benzothiadiazides: Cyclothiazide;

Benzoylpiperidines: CX516, CX546;

Biarylopropylsulfonamides: LY392098, LY404187, LY451646;

Metabotropic glutamate receptors (mGluRs): 2-methyl-6-(phenylethynyl)-pyridine (MPEP), 3-[(2-methyl-1,3-thiazol-4-yl)ethynyl]-pyridine (MTEP), JNJ16259685, CPCOOEt, MGS0039, LY341495, LY354740, ACPT-1/L-SOP (L-serine-O-phosphate), HomoAMPA, N-pheynl-7-(hydroxyimino) cyclopropa[b]chromen-1a-carboxamide;

GABA antagonists: CGP36742, CGP56433, CGP56999;

NK1 antagonists: GW823296, GW679769, GW597599 (Vestipitant), R673, CP-122,721, L-759274, GR205171, L733060;

NK2 antagonists: SR48968;

CRF1 antagonists: DMP696, DMP904, GW876008, AAG561, TS-041, CP-154,526 (antalarmin), SSR125543, R278995/CRA0450, R121919;

Arginine vasopressin V1b antagonists: SSR149415;

MCH receptor antagonists: T-226296.

In some patients, it reportedly is useful to augment antidepressant treatment with lithium or triiodothyronine.

Thus, in another illustrative embodiment, the invention comprises a kit comprising one or more pharmaceutical dosage units of MA-1 or MA-2 and one or more pharmaceutical dosage units of a antidepressant, wherein either or both of MA-1 or MA-2 unit dose form and the antidepressant unit dose form can also comprise, respectively, an antidepressant or an anti-psychotic, and optionally, one or more additional pharmaceutically active ingredients. In another embodiment, the invention comprises administering MA-1 or MA-2 and the other agent or agents at different time intervals, such that an effective amount of each is maintained in the patient's bloodstream in the appropriate amounts at the appropriate times. Such kit could facilitate, e.g., administration of MA-1 or MA-2 to be taken at different time intervals than the other agent or agents. In a related embodiment, the kit comprises pharmaceutical dosage units of one agent alone and other pharmaceutical dosage units comprising both agents. In this way, for example, MA-1 or MA-2 could be taken alone during the day and with the other agent or agents in the evening.

When used in such combinations, the dose of each agent is expected to be approximately the same as, or less than, an effective amount of either alone. For example, each pharmaceutically active ingredient can be administered in doses that are about 20% to about 80% of the dose in which each ingredient would be administered alone.

The two (or more) agents can be administered more or less simultaneously, i.e., concomitantly (e.g., within about 0 to about 5 minutes of each other, preferably within about a minute apart, or they can be administered at different times. For example, in one aspect, the invention is a pharmaceutical composition comprising both the anti-psychotic agent and the other agent or agents. This embodiment, for example, comprises a pill or capsule having both active pharmaceutical ingredients either admixed together or having each active pharmaceutical ingredient in a discrete portion of the pill or capsule.

Unit dose forms of the invention, whether they comprise MA-1 or MA-2 or an active metabolite thereof as the sole active pharmaceutical ingredient or in combination with another agent, e.g., an antipsychotic or antidepressant, can also be formulated in a controlled release form, e.g., delayed, sustained, or pulsatile release. With such form, in the case of combinations, MA-1 or MA-2 or active metabolite thereof can be released at the same or different rates and times as the other agent or agents.

### EXAMPLES

The examples that follow are illustrative and not limiting of the invention and illustrate the usefulness of MA-1 in the prevention and treatment of symptoms of depressive disorders.

### Examples 1-3

MA-1 was tested in the following 3 models: (1) stress-induced cGMP elevation, (2) mouse Forced Swim test and (3) rat Forced Swim test. Below are the protocols used and results obtained from these studies.

Stress-Induced Cerebellar cGMP Elevation

Protocol: Animals were placed into a shock chamber with a steel grid floor and shocked at 1 mA for 10 seconds. One minute following the stressor, the animals were placed into a plastic restraint tube and sacrificed by microwave irradiation (1.8 sec at 3.5 kW). The cerebellum was rapidly removed, snap frozen, and stored at −80° C. prior to the cGMP assay. Non-stressed animals were taken directly from their cages and sacrificed by microwave irradiation and tissues were processed in a similar manner. Drug dosing was performed 30-60 min prior to foot-shock stress. For the cGMP assay, the tissue was homogenized in 2 ml of 1% perchloric acid using a Brinkman Polytron at setting #5 for ˜15 sec each and placed on ice until all samples were homogenized. Samples were then placed in an 85C water bath for 5 min, centrifuged at 2500G for 15 min, and ˜0.5 ml of the supernatant was collected for analysis. Supernatants were diluted 1:20 in sodium acetate buffer according to the directions of the manufacturer of the 1251-cGMP flashplates. Diluted samples were incubated overnight in flashplate wells with 1251-cGMP, assayed on a gamma-counter plate reader, and converted to pmol cGMP/mg tissue using a standard curve generated in the same experiment.

Results: Rats receiving an electric shock showed ˜2.5× increase in cerebellar cGMP levels. This increase was attenuated ˜50% by treatment with MA-1 at doses of 0.1-10 mg/kg. Although the effect appeared to be maximal without dose-responsiveness, lower doses were not tried.

### Mouse Forced Swim Test

Protocol: Animals were maintained on a 12:12 LD cycle with lights on at 0600 h. Mice were placed into the testing room at least 1 h prior to the start of the test. Vehicle, amitriptyline and MA-1 were administered under one of three conditions: A) acute treatment, animals dosed 30 minutes prior to testing; B) 4 day subchronic AM treatment, with dosing occurring during the early morning period (0900-1100 h), with the final dose occurring 30 min prior to testing; and C) subchronic PM treatment with dosing occurring during the evening period (1730-1800 h, right before lights off), and the forced swim test took place the following morning. Animals were tested in the forced swim test using a modification of the protocol originally described by Porsolt et al. (1978). Mice were placed into 1 L beakers (KIMAX #14005) filled with 800 ml of water (20-22° C.) for a 7 min swim period. Animals were only scored for the last 5 minutes of the test and were assigned either a “0” if they were actively swimming or “1” if they were immobile, except for small movements needed to keep afloat. During the 5 minute scoring period, there are ten 30 sec intervals scored for a total possible score of 0-10 for each mouse. Data was reported as median (interquartile range). Each study was run independently with separate groups of naive mice. Data were analyzed using Statview (SAS, Cary, N.C.) with a Kruskal-Wallis analysis, followed by Mann-Whitney U-test with the significance level set at p<0.05.

Results: MA-1 was tested for efficacy in the mouse forced swim model under three conditions including (A) acute treatment, with testing 30 minute post-dose, (B) 4-day sub-chronic treatment with AM dosing and testing 30 minutes following the final dose and (C) 4-day sub-chronic treatment with PM dosing and testing the following morning. Amitriptyline was used as a positive control in this assay, and was active under conditions A and B, but did not show activity under condition C. However, MA-1 did not demonstrate activity in this assay under any of the conditions tested.

### Rat Forced Swim Test

Protocol: Animals were tested in the forced swim test using the protocol originally described by Porsolt et al (Eur. J. Pharmacol., 47, 379-391,1978). Rats were individually placed in a cylinder (Height=40 cm, Diameter=20 cm) containing 13 cm water (25° C.) for 15 minutes on the first day of the experiment (Session 1) and were then put back in the water 24 hours later for a 5 minute test (Session 2). The duration of immobility during the 5 minute test was measured. Six rats were studied per group. The test was performed blind. Session 1 and Session 2 were performed either during the light cycle, i.e. between 2.5 and 5.5 hours after lights-on, or during the dark cycle, i.e. between 2.5 and 5.5 hours after lights-off. The tests during the light cycle were therefore performed between 9:30 am and 12:30 pm, whereas the tests during the dark cycle, because of the light cycle shift, were performed between 14:30 pm and 17:30 pm.

To permit the 2 phases of the experiment (light phase and dark phase) to be performed on the same day by the same laboratory technician, the animals to be tested during the dark phase were submitted to a light cycle shift 12 days prior to the first session of the forced swim test whereby the light/dark cycle was advanced 7 hours (lights-on: 0:00 am, lights-off: 12:00 pm). The 12-day period was estimated to be sufficient for the dark-cycle animals to adjust to the shift. To habituate the rats to the light cycle shift, the dark-cycle animals were submitted to the shift 12 days prior to Session 1. To ensure otherwise similar conditions between the light-cycle and dark-cycle animals, all animals to be used in the experiment were from the same delivery batch and were placed in their experimental living cages at the same time, i.e. 12 days before Session 1.

Testing during the light phase was performed under normal laboratory illumination, and testing during the dark phase was performed under infrared illumination. MA-1, agomelatine, and melatonin were evaluated at 2 oral (p.o.) doses each, administered twice (24 hours and 1 hour before Session 2). The first administration was given immediately after Session 1. Imipramine (64 mg/kg p.o.), administered twice under the same experimental conditions, was used as reference substance.

Result: Rats were dosed and tested during either the dark phase (table 1) or the light phase (table 2) of the 24 hr cycle, to investigate the potential for a sensitivity to circadian time. Compounds tested included imipramine as a positive control (64 mg/kg), melatonin (10 and 50 mg/kg), agomelatine (10 and 50 mg/kg) and MA-1 (1 and 10 mg/kg). Doses were chosen to coincide with the range where activity has been reported in the literature for this or other behavioral assays. Activity was more robust during the dark phase for all melatonin agonists, with agomelatine showing a 60% and 33% decrease in immobility time at 10 and 50 mg/kg respectively. MA-1 also showed a significant decrease in immobility time at both doses tested, with a 37% and 41% decrease in immobility seem at 1 and 10 mg/kg respectively. Activity was also observed in animals tested during the light phase (table 2), although the effects were more modest and less consistent across doses tested.

TABLE 1
EFFECTS OF AGOMELATINE, MA-1 MELATONIN AND
IMIPRAMINE IN THE BEHAVIORAL DESPAIR TEST (DARK
CYCLE) IN THE RAT (6 RATS PER GROUP)

| | |
|---|---|
| TREATMENT | |
| (mg/kg) | DURATION OF IMMOBILITY (s) |

| p.o. −24 h | | p | % change |
|---|---|---|---|
| and −60 min | mean ± s.e.m. | value | from control |
| Vehicle #1 | 210.0 ± 5.6 | — | — |
| agomelatine (10) | 84.8 ± 8.2 *** | <0.0001 | −60% |
| agomelatine (50) | 140.2 ± 19.1 * | 0.0107 | −33% |
| MA-1 (1) | 133.0 ± 6.6 *** | <0.0001 | −37% |
| MA-1 (10) #2 | 124.8 ± 18.8 ** | 0.0024 | −41% |
| Melatonin (10) | 132.8 ± 16.5 ** | 0.0028 | −37% |
| Melatonin (50) | 166.8 ± 16.6 * | 0.0492 | −21% |
| Imipramine (64) | 63.0 ± 11.1 *** | <0.0001 | −70% |

Student's t test:
* = p < 0.05;
** = p < 0.01;
*** = p < 0.001
#1: escape (1/6).
#2: dead (1/6).

TABLE 2
EFFECTS OF AGOMELATINE, MA-1, MELATONIN AND
IMIPRAMINE IN THE BEHAVIORAL DESPAIR TEST (LIGHT
CYCLE) IN THE RAT (6 RATS PER GROUP)

| | |
|---|---|
| TREATMENT | |
| (mg/kg) | DURATION OF IMMOBILITY (s) |

| p.o. −24 h | | p | % change |
|---|---|---|---|
| and −60 min | mean ± s.e.m. | value | from control |
| Vehicle | 168.3 ± 14.1 | — | — |
| Agomelatine (10) | 97.8 ± 13.5 ** | 0.0047 | −42% |
| Agomelatine (50) | 191.0 ± 9.4 NS | 0.2114 | +13% |
| MA-1 (1) | 145.3 ± 26.0 NS | 0.4548 | −14% |
| MA-1 (10) | 126.3 ± 26.9 NS | 0.1967 | −25% |
| Melatonin (10) | 101.3 ± 18.8 * | 0.0172 | −40% |
| Melatonin (50) | 167.7 ± 10.8 NS | 0.9709 | 0% |
| Imipramine (64) | 49.5 ± 10.6 *** | <0.0001 | −71% |

Student's t test:
NS = Not Significant;
* = p < 0.05;
** = p < 0.01;
*** = p < 0.001

Conclusions: This set of studies was designed to test whether MA-1 showed similar activity to other melatonin agonists in rodent behavioral models of stress and behavioral despair. In those models in which other melatonin agonists showed activity, MA-1 was active. Melatonin and agomelatine have previously shown activity in the stress-induced cGMP assay (data not shown) at levels similar to what was observed for MA-1. In addition, MA-1 showed activity in the rat FST (Porsolt labs) similar in magnitude to that shown by agomelatine and melatonin. Although MA-1 was not active in the mouse FST, we have not shown activity for other melatonin agonists in this assay. The lack of effect in the mouse FST as compared to the rat assay run by Porsolt Labs, is not simply due to a species difference since we have also not observed activity for melatonin agonists in another version of the rat FST. This suggests that subtle differences in assay design, route of administration, or time of dosing, may be critical for melatonin agonists to work in this assay. In yet another study not reported here, MA-1 tested in rats in a modified forced swim test at 5 mg/kg and 10 mg/kg and did not show effects on immobility, swimming, or climbing that were statistically different from vehicle.

1. A method for treating major depression in a human comprising internally administering to the human an effective amount of MA-1.

2. The method of claim 1, wherein the major depression includes at least one symptom selected from a group consisting of: persistent sad, anxious, or empty mood; feelings of hopelessness; pessimism; feelings of guilt, worthlessness, or helplessness; loss of interest or pleasure in hobbies and activities that were once enjoyed, including sex; decreased energy, fatigue, or being slowed down; difficulty concentrating, remembering, or making decisions; insomnia, early-morning awakening, or oversleeping; appetite and/or weight loss or overeating and weight gain; thoughts of death or suicide; suicide attempts; restlessness; irritability; persistent physical symptoms that do not respond to treatment, such as headaches, digestive disorders, and chronic pain; or any combination of the preceding.

3. The method of claim 1, which further comprises administering a second antidepressant medication.
